(19) United States
(12) Patent Application Publication
(10) Pub. No.: US 2019/0218315 A1
(43) Pub. Date: Jul. 18, 2019

(54) ANIONIC SURFACTANT AND USE THEREOF

(71) Applicant: RHODIA OPERATIONS, Paris (FR)

(72) Inventors: Mingxing HUANG, Singapore (SG); Peng XU, Singapore (SG); Zhihua ZHANG, Singapore (SG); Man CHEN, Singapore (SG)

(21) Appl. No.: 16/334,438

(22) PCT Filed: Sep. 21, 2017
(86) PCT No.: PCT/CN2017/102583

§ 371 Date: Mar. 19, 2019

(30) Foreign Application Priority Data
Sep. 22, 2016 (CN) PCT/CN2016/099671

(51) Int. Cl. C08F 2/26; C07C 309/10

(52) U.S. Cl. CPC C08F 2/26; C07C 309/10

(57) ABSTRACT

A compound of formula (I): R₁O—(CH₂CH₂O)ₓ—(CH₂CH(R₂)—O)_{y}—SO₃M, wherein R₁, R₂, x, y and M are defined herein. A composition comprising the compound of formula (I) and the use thereof.

This application claims priority to PCT international patent application no. PCT/CN2016/099671 filed on Sep. 22, 2016, the whole content of this application being incorporated herein by reference for all purposes.

### TECHNICAL FIELD

The present invention relates to a compound which may be used for emulsion polymerization. The compound is notably an anionic surfactant.

### BACKGROUND ART

Emulsion polymerization is the most important industrial method for manufacturing of aqueous dispersion polymers, and it plays significant role in the industry of construction, adhesive, textile, paper, ink and so forth. Emulsion polymerization is typically performed in an aqueous medium in the presence of a surfactant and a water-soluble initiator and usually rapidly produces high molecular weight homo- or copolymers at high solids content and low dispersion viscosity. Its application requires the emulsification of the monomer in a medium, usually water, through the use of surfactants.

The final product resulting from emulsion polymerization is normally an opaque, grey or milky-white dispersion of high molecular weight polymer(s). Such dispersions can be used for adhesives, binders for fibres and particulate matter, protective and decorative coatings, dipped goods, foam, paper coatings, backings for carpet and upholstery, modifiers for bitumen and concrete and thread and textile modifiers, biomedical applications as protein immobilisers, visual detectors in immunoassays, as release agents, in electronic applications as photoresists for circuit boards, in batteries, conductive paint, copy machines, and as key components in molecular electronic devices.

Surfactants play an important role in the emulsion polymerization. A number of surfactants have been used in emulsion polymerization. For example, PCT international patent publication no. WO 2010072029 A1 discloses an alkoxylated alcohol non-ionic surfactant for use as an emulsifier in emulsion polymerization. US patent publication no. 2012/0136119 A1 discloses anionic and non-ionic styrenated phenol ethoxylates for use in emulsion polymerization process. Nevertheless, due to the nature of surfactant, the use of surfactants also brings some problems to end users, such as foaming problem in emulsion polymerization process and paint formulation, compromised wet scrub resistance of paint, etc. Thus, one objective is to provide a surfactant for use in emulsion polymerization process which has good efficiency. It is also an objective that the surfactant possesses some additional advantage besides their conventional role such as emulsifying monomer and stabilizing the resultant polymer emulsion.

### SUMMARY OF INVENTION

It has been surprisingly found that the above objectives can be met by the present invention.

In one aspect, the present invention provides a compound of formula (I):

R₁O—(CH₂CH₂O)ₓ—(CH₂CH(R₂)—O)_{y}—SO₃M (I)

wherein R₁ is a linear or branched, saturated or unsaturated, C₄-C₁₈ hydrocarbon group;

R₂ is CH₃ or CH₂CH₃;

x is a real number in the range of from 1 to 11;

y is a real number in the range of from 1 to 20; and

M is a cation.

Notably, the present invention provides a compound of formula (I):

R₁O—(CH₂CH₂O)ₓ—(CH₂CH(R₂)—O)_{y}—SO₃M (I)

wherein R₁ is a linear or branched C₁₂-C₁₈ alkyl group;

R₂ is CH₃ or CH₂CH₃;

x is a real number in the range of from 3 to 8;

y is a real number in the range of from 3 to 8; and

M is a cation.

The present invention further provides a composition comprising said compound of formula (I).

Said composition may further comprise a compound of formula (II):

R₁O—(CH₂CH₂O)ₓ—(CH₂CH(R₂)—O)_{y}—H (II)

wherein R₁ is a linear or branched, saturated or unsaturated, C₄-C₁₈ hydrocarbon group;

R₂ is CH₃ or CH₂CH₃;

x is a real number in the range of from 1 to 11;

y is a real number in the range of from 1 to 20.

Preferably, said composition further comprises a co-surfactant.

Preferably, said composition further comprises water.

In another aspect, the present invention provides a method for the emulsion polymerization of at least one ethylentically unsaturated monomer, containing at least one carbon-to-carbon double bond, said method comprising polymerizing said ethylentically unsaturated monomer in an aqueous medium in the presence of the compound of formula (I) or said composition, and a water-soluble initiator.

In still another aspect, the present invention provides a use of the compound of formula (I) or said composition for emulsion polymerization.

### DETAILED DESCRIPTION

Throughout the description, including the claims, the term “comprising one” or “comprising a” should be understood as being synonymous with the term “comprising at least one”, unless otherwise specified, and “between” should be understood as being inclusive of the limits.

It should be noted that in specifying any range of concentration, weight ratio or amount, any particular upper concentration, weight ratio or amount can be associated with any particular lower concentration, weight ratio or amount, respectively.

As used herein, the term “alkyl” means a saturated hydrocarbon radical, which may be straight, branched or cyclic, such as methyl, ethyl, n-propyl, iso-propyl, n-butyl, sec-butyl, t-butyl, pentyl, n-hexyl, cyclohexyl.

As used herein, the term “hydroxyalkyl” means an alkyl radical, which is substituted with a hydroxyl groups, such as hydroxymethyl, hydroxyethyl, hydroxypropyl, and hydroxydecyl.

As used herein, the terminology “Cₘ-Cₙ” in reference to an organic group, wherein m and n are each integers, indicates that the group may contain from m carbon atoms to n carbon atoms per group.

The present invention provides a compound of formula (I):

R₁O—(CH₂CH₂O)ₓ—(CH₂CH(R₂)—O)_{y}—SO₃M (I)

wherein R₁ is a linear or branched, saturated or unsaturated, C₄-C₁₈ hydrocarbon group;

R₂ is CH₃ or CH₂CH₃;

x is a real number in the range of from 1 to 11;

y is a real number in the range of from 1 to 20; and

M is a cation.

In formula (I), R₁, for example, may be a C₄-C₁₈ alkyl, C₄-C₁₈ alkenyl or C₄-C₁₈ hydroxylalkyl group, preferably, a C₄-C₁₈ alkyl group. R₁ may be linear or branched. For example, R₁ may be a C₄ alkyl, C₄ alkenyl or C₄ hydroxylalkyl group, a C₈ alkyl, C₈ alkenyl or C₈ hydroxylalkyl group, a C₁₀ alkyl, C₁₀ alkenyl or C₁₀ hydroxylalkyl group, a C₁₂ alkyl, C₁₂ alkenyl or C₁₂ hydroxylalkyl group, a C₁₄ alkyl, C₁₄ alkenyl or C₁₄ hydroxylalkyl group, a C₁₆-C₁₈ alkyl, C₁₆-C₁₈ alkenyl or C₁₆-C₁₈ hydroxylalkyl group.

According to any one of the invention embodiments, R₁ may preferably be a C₁₂-C₁₈ alkyl, C₁₂-C₁₈ alkenyl or C₁₂-C₁₈ hydroxylalkyl group, more preferably, a C₁₂-C₁₈ alkyl group.

Examples of the R₁ group include but are not limited to: octyl, nonyl, decyl, undecyl, lauryl, tridecyl, cetyl, palmityl, stearyl and oleyl group.

R₂ is CH₃ or CH₂CH₃, preferably CH₃.

It is appreciated that x refers to the average degree of ethoxylation and y refers to the average degrees of propoxylation and/or butoxylation (depending on the identity of R₂). Thus, x and y need not to be integers. Taken together, x and y establish a degree of alkoxylation in an oligomer distribution. It is also appreciated that the EO portion and the PO/BO portion in the compound of formula (I) are the result of a block feed wherein addition of the EO portion is firstly carried out, followed by addition of the PO/BO portion.

Preferably, x is a real number in the range of 1 to 8, more preferably, in the range of 3 to 8.

Preferably, y is a real number in the range of 1 to 11, more preferably, in the range of 3 to 8.

M, for example, may be Na⁺, K⁺, Li⁺ or —(HNR₃R₄R₅)⁺ wherein R₃, R₄ and R₅ are independently H, a C₁-C₆ alkyl or C₁-C₆ hydroxylalkyl group.

Examples of the compound of formula (I) include but are not limited to: decyl alkoxylated sulfate, ammonium salt; decyl alkoxylated sulfate, sodium salt; decyl alkoxylated sulfate, potassium salt; lauryl alkoxylated sulfate, ammonium salt; lauryl alkoxylated sulfate, sodium salt; lauryl alkoxylated sulfate, potassium salt; tridecyl alkoxylated sulfate, ammonium salt; tridecyl alkoxylated sulfate, sodium salt; tridecyl alkoxylated sulfate, potassium salt.

In another aspect, the present invention provides a composition comprising the compound of formula (I) described herein.

Typically, the compound of formula (I) has the structure of an anionic surfactant. Accordingly, said composition is a surfactant composition and such surfactant composition can notably be utilized in emulsion polymerization. Surfactants are compounds being able to reduce the surface tension between two liquids or between a liquid and a solid. Surfactants are amphiphilic materials, and they contain both hydrophobic groups and hydrophilic groups. The compound of formula (I) can be highly efficient in emulsion polymerization. In the meantime, the compound may provide the emulsion dispersion that is formed with additional excellent properties such as low foam property, improved freeze-thaw stability and good Ca²⁺ stability.

According to any one of the invention embodiments, the composition may further comprise a compound of formula (II):

R₁O—(CH₂CH₂O)ₓ—(CH₂CH(R₂)—O)_{y}—H (II)

wherein R₁ is a linear or branched, saturated or unsaturated, C₄-C₁₈ hydrocarbon group;

R₂ is CH₃ or CH₂CH₃;

x is a real number in the range of from 1 to 11;

y is a real number in the range of from 1 to 20.

In formula (II), R₁, for example, may be a C₄-C₁₈ alkyl, C₄-C₁₈ alkenyl or C₄-C₁₈ hydroxylalkyl group, preferably, a C₄-C₁₈ alkyl group. R₁ may be linear or branched. For example, R₁ may be a C₄ alkyl, C₄ alkenyl or C₄ hydroxylalkyl group, a C₈ alkyl, C₈ alkenyl or C₈ hydroxylalkyl group, a C₁₀ alkyl, C₁₀ alkenyl or C₁₀ hydroxylalkyl group, a C₁₂ alkyl, C₁₂ alkenyl or C₁₂ hydroxylalkyl group, a C₁₄ alkyl, C₁₄ alkenyl or C₁₄ hydroxylalkyl group, a C₁₆-C₁₈ alkyl, C₁₆-C₁₈ alkenyl or C₁₆-C₁₈ hydroxylalkyl group.

R₁ is preferably a C₁₂-C₁₈ alkyl, C₁₂-C₁₈ alkenyl or C₁₂-C₁₈ hydroxylalkyl group, more preferably, a C₁₂-C₁₈ alkyl group.

Examples of the R₁ group include but are not limited to: octyl, nonyl, decyl, undecyl, lauryl, tridecyl, cetyl, palmityl, stearyl and oleyl group.

R₂ is CH₃ or CH₂CH₃, preferably CH₃.

Preferably, x is a real number in the range of 1 to 8, more preferably, in the range of 3 to 8.

Preferably, y is a real number in the range of 1 to 11, more preferably, in the range of 3 to 8.

x and y as defined in formula (II) represent the average degree of alkoxylation and should be construed according to the way in the definition of formula (I).

When the composition comprises a compound of formula (II) in addition to the compound of formula (I), the groups R₁ and R₂, x and y in formula (I) and formula (II) may be the same or different. In some embodiments, the groups R₁ and R₂, x and y in formula (I) and formula (II) are the same.

The composition may further include additional components such as water, co-surfactants, amine oxides, alkyl amine oxides, solvents, chelating agents, bases such as monoethanolamine, diethanolamine, triethanolamine, potassium hydroxide, sodium hydroxide, or other bases, and other conventional formulation ingredients. Advantageously, the composition comprises water.

The co-surfactants suitable for the present invention include but are not limited to: ethoxylated alcohol and the salts thereof, sodium alkylbenzene sulfonates, alkyldiphenyloxide disulfonates, ethoxylated alkylphenol sulfates and phosphates, alkyl sulfosuccinates, and sulfates and phosphates of fatty alcohols, alkylphenol ethoxylates, particularly ethoxylated alcohol. Examples of suitable co-surfactant include RHODASUF® BC-8509, RHODAPEX® LA-40S and ABEX® AP-470Z available from the Solvay Company. When used in combination with the co-surfactants, the ratios are not limited but are also dictated by the desired emulsion properties.

The compound of formula (I) may be present in an amount of from 10 wt % to 90 wt %, preferably from 30 wt % to 60 wt %; based on the total weight of the composition. The compound of formula (II) may be present in an amount of from 0.1 wt % to 30 wt %, preferably from 0.1 wt % to 10 wt % by weight; based on the total weight of the composition.

In embodiments wherein the composition comprises both the compound of formula (I) and the compound of formula (II), the weight ratio of the compound of formula (I) to the compound of formula (II) may be from 99:1 to 10:90. Preferably, the weight ratio is from 95:5 to 50:50, more preferably, from 90:10 to 70:30.

The group R₁ as defined in formula (I) and formula (II) are typically derived from alcohols. The alcohol used as source for obtaining the compound of formula (I) and (II) can be a single alcohol or blend. Examples of the alcohols used include octanol, nonanol, decanol, undecanol, dodecanol, tridecanol.

The compound of formula (II) may be purchased from commercial vendors or they may be prepared by those skilled in the art. In a typical procedure, a suitable alcohol or fatty acid alcohol is alkoxylated with alkylene oxide compounds. Alkoxylation processes may, for instance, be carried out in the presence of acidic or alkaline catalysts, or by using metal cyanide catalysts. Alkaline catalysts may include, for instance, hydroxides or alcoholates of sodium or potassium, including NaOH, KOH, sodium methoxide, potassium methoxide, sodium ethoxide and potassium ethoxide. Base catalysts are normally used in a concentration of from 0.05% to about 5% by weight, preferably about 0.1% to about 1% by weight based on starting material.

The addition of alkylene oxides may, for instance, be carried out in an autoclave under pressures from about 10 psig to about 200 psig, preferably from about 60 to about 100 psig. The temperature of alkoxylation may range from about 30° C. to about 200° C., preferably from about 100° C. to about 160° C. After completion of oxide feeds, the product is typically allowed to react until the residual oxide is less than about 10 ppm. After cooling the reactor to an appropriate temperature ranging from about 20° C. to 130° C., the residual catalyst may be left un-neutralized, or neutralized with organic acids, such as acetic, propionic, or citric acid. Alternatively, the product may be neutralized with inorganic acids, such as phosphoric acid or carbon dioxide. Residual catalyst may also be removed using ion exchange or an adsorption media, such as diatomaceous earth.

The compound of formula (I) may be prepared by the sulfation of the compound of formula (II) using a catalyzed sulfamic acid sulfation process. Typically, pre-heated nonionic alkyl alkoxylate (50° C.) may be first mixed well with sulfamic acid and dicyandiamide. When the mixture is homogenized by mechanical stirring and heated to higher temperature, preferably from 100° C. to 130° C., under nitrogen protection, the sulfation reaction between alkyl alkoxylate and sulfamic acid will occur in the presence of the catalyst dicyandiamide to produce the corresponding alkyl alkoxylate sulfate. The product can be collected when it is cooled down to ambient temperature. The major component of the product may be alkyl alkoxyalte sulfate, or the mixture of alkyl alkoxylate sulfate and nonionic alkyl alkoxylate, depending on the conversion rate of the reaction. The collected product may be acidic, and it may be neutralized by ammonia solution to afford a neutral product.

The composition of the invention may be used as an emulsifier for aqueous emulsions or dispersions of polymers and/or copolymers which are normally obtainable by emulsion polymerization. In one aspect, the present invention provides a method for the emulsion polymerization of at least one ethylentically unsaturated monomer, containing at least one carbon-to-carbon double bond, said method comprising polymerizing said ethylentically unsaturated monomer in an aqueous medium in the presence of the compound of formula (I) or the composition described herein, and a water-soluble initiator. The resulting aqueous emulsions or dispersions can be used for adhesives, binders for fibres and particulate matter, protective and decorative coatings, dipped goods, foam, paper coatings, backings for carpet and upholstery, modifiers for bitumens and concrete and thread and textile modifiers. The resulting aqueous emulsions or dispersions can also be used, for biomedical applications as protein immobilisers, for visual detectors in immunoassays as release agents, in electronic applications as photoresists for circuit boards, in batteries, conductive paint, copy machines, and as key components in molecular electronic devices.

There are no particular restrictions as to the nature of the polymers and copolymers. Polymers or copolymers based on the following monomer units are preferred: acrylic acid, acrylates, butadiene, methacrylic acid, methacrylates, styrene, ethylene and vinyl acetate, acrylamide, acrylonitrile.

Suitable monomers that may be polymerized by the practice of the present invention include numerous ethylenically unsaturated monomers such as vinyl monomers or acrylic monomers. Typical vinyl monomers suitable for use in accordance with the present invention include, but are not limited to, vinyl esters such as vinyl acetate, vinyl propionate, vinyl benzoate, etc; vinyl aromatic hydrocarbons such as styrene, methyl styrenes, other vinyl aromatics such as vinyl toluenes, vinyl napthalenes, divinyl benzene, etc. Halogenated vinyl monomers such as vinyl chloride, vinylidene chloride, etc. may also be used.

Suitable acrylic monomers which may be used in accordance with the present invention comprise compounds with acrylic functionality such as alkyl acrylates and methacrylates, acrylate acids and methacrylate acids as well as acrylamides and acrylonitrile. Typical acrylic monomers include, but are not limited to methyl acrylate and methyl methacrylate, ethyl, propyl, and butyl acrylate and methacrylate, benzyl acrylate and methacrylate, cyclohexyl acrylate and methacrylate, decyl and dodecyl acrylate and methacrylate, etc. Other typical acrylic monomers include hydroxy alkyl acrylates and methacrylates such as hydroxypropyl and hydroxyethyl acrylate and methacrylate, acrylic acids such as methacrylic and acrylic acid, and amino acrylates and methacrylates. It will be recognized by those familiar with the art that other unsaturated monomers which are suitable for free radical addition polymerization may also be used in accordance with the present invention.

Numerous free radical forming compounds may be used as the water-soluble initiator utilized as catalysts in the method of the invention. Typical compounds used as catalysts may be those that form free radicals via thermal decomposition, referred to in the art as “thermal initiators” or combinations of compounds that fond free radicals via oxidation/reduction reactions. Such catalysts are combinations of an oxidizing agent and a reducing agent and are commonly referred to in the art as “redox initiators”. Either thermal or redox catalysts may be used in the method of the present invention.

Typical catalysts utilized as the thermal initiators include, for example, persulfates, specifically potassium persulfate, sodium persulfate, ammonium persulfate and the like. Typical redox initiators include, for example, combinations of oxidizing agents or initiators such as peroxides, specifically benzoyl peroxide, t-butyl hydroperoxide, lauryl peroxide, hydrogen peroxide, 2,2′-diazobisisobutyronitrile, and the like. Typical reducing agents include sodium bisulfite, sodium formaldehyde sulfoxylate, sodium hydrosulfite, and ascorbic and isoascorbic acid. The water-soluble initiator may be employed in an amount of from 0.1 to 3 weight percent of the total monomer weight, and more preferably from about 0.1 to 1 weight percent of the total monomer charge.

Other additives or components which are known to those skilled in the art may also be used in accordance with the present invention. These include chain transfer agents, which are used to control molecular weight, additives to adjust pH, and compounds utilized as protective colloids which provide additional stability to the latex particles.

Any of the conventional methods employed in the emulsion polymerization process may also be used in accordance with the present invention. These include both standard and pre-emulsion monomer addition techniques as well as staged monomer addition.

A person skilled in the art can readily determine the effective amount of the composition of the invention that should be used in an emulsion polymerization formulation, via a combination of general knowledge of the applicable field as well as routine experimentation where needed. For instance, in some aspects, a quantity of from 0.01% to 10% BOTM (based on total monomer) by active weight of composition, preferably from 0.1 to 5% BOTM by active weight of the composition, more preferably from 0.2 to 3% BOTM by active weight of the composition, based on the total weight of monomers used in the emulsion polymerization, may be suitable.

In another aspect, the present invention also provides a use of the compound of formula (I) or the composition described herein for emulsion polymerization. Notably, the present invention provides a use of the compound of formula (I) or the composition described herein as an emulsifier for emulsion polymerization.

Should the disclosure of any patents, patent applications, and publications which are incorporated herein by reference conflict with the description of the present application to the extent that it may render a term unclear, the present description shall take precedence.

### EXAMPLES

### Materials

Anionic Surfactant 1: alkyl alkoxylate sulfate (solid 60%) having the formula: C₁₂H₂₅O-(EO)₄(PO)₄—SO₃NH₄;

Anionic Surfactant 2: alkyl alkoxylate sulfate (solid 60%) having the formula: C₁₂H₂₅O-(EO)₄(PO)₄—SO₃Na;

Anionic Surfactant 3: alkyl alkoxylate sulfate (solid 60%) having the formula: C₁₂H₂₅O—(PO)₄(EO)₄—SO₃Na;

RHODASUF® BC-8509: ethoxylated tridecanol, available from the Solvay Company;

ABEX® AP-470Z: mixture of ethoxylated alcohols, available from the Solvay Company;

RHODAPEX® LA-40S: sodium salt of sulphated alcohol ethoxylate, available from the Solvay Company;

SIPOMER® COPS-1: a solution of anionic compound, available from the Solvay Company;

The following abbreviation of monomers is used:

S: styrene

BA: butyl acrylate

MMA: methyl methacrylate

MAA: methacrylic acid

AM: acrylamide

APS: ammonium persulfate

SPS: sodium persulfate

TBP: di-tert-butyl hydroperoxide (70% solution)

IAA: D-isoascorbic acid

PME: pre-monomer emulsion Monomers:

S: styrene

BA: butyl acrylate

MMA: methyl methacrylate

MAA: methacrylic acid

AM: acrylamide

APS: ammonium persulfate

SPS: sodium persulfate

TBP: di-tert-butyl hydroperoxide (70% solution)

IAA: D-isoascorbic acid

PME: pre-monomer emulsion

### Example 1: Synthesis of the Anionic Surfactants

Anionic Surfactant 1 was prepared through the procedures described below. 186 g lauryl alcohol were mixed with 0.3 g 50% KOH solution, and then dried under a vacuum at 90° C. for about 30 minutes. 176 g ethylene oxide were fed into the autoclave at the feed rate of about 5.0 g per minute. After a suitable cookout at 130° C., the resultant intermediate was further propoxylated by feeding 232 g propylene oxide. After a suitable cookout at 130° C., the material was removed from the reactor and neutralized with acetic acid to a pH range of 5-7 (as a 10 wt % aqueous solution) to afford the alkyl alkoxylate.

120.0 g of the alkyl alkoxylate were charged into a three-necked flask, which was equipped with mechanical stirrer, thermometer and Schlenk line, and then heated to 50° C. under stirring. 0.62 g dicyandiamide was then added into the flask. After 10 minute mixing, 17.03 g of sulfamic acid were then added. Purged the system with nitrogen, and then heated it slowly to 120° C. After 2 hour reaction, cooled down the reactor to 50° C., and collected the product. The product was neutralized with 25 wt % ammonia solution to pH 7, and diluted with deionized water to a desired concentration.

In one typical synthesis, the final product was collected as clear solution with pale yellow color. The product had a solid content of 61.01%. The successful sulfation was revealed by a Hyamine titration, from which the content of the anionic surfactant in product solution was determined as 58.12%.

The collected product was characterized by NMR spectroscopy. The formation of the desired compound from the sulfation reaction was confirmed by the ¹³C NMR characterization. The ¹³C NMR spectrum showed the disappearance of —CH₂OH carbon at the chemical shift at δ=61.39 ppm and the presence of —CH₂OSO₃⁻ carbon at δ=71.46 ppm. The structure of the collected product was further consolidated by the ¹H NMR analysis. In the ¹H NMR spectrum, the most significant signal was at 4.14 ppm, which was from the methylene group next to sulfate functional group, indicating the formation of sulfate after the sulfation reaction. All other signals can be allocated to the produced alkyl alkoxylated sulfate.

Anionic Surfactant 2 was prepared by the procedures described below. 120.0 g of alkyl alkoxylate were charged into a flask and stirred. Next, an air-SO₃ gas stream containing about 3.5 wt % of sulphur trioxide in dried air was introduced via gas sparger. Cool water batch was employed throughout the reaction to maintain the temperature of the reaction mixture in the range of 38-45° C. When the acid number of the reaction mixture was within the desired range, i.e. 80-90 mg KOH/g, addition of air-SO₃ was stopped. Nitrogen purge was then applied for 15-20 minutes to remove the sulphur trioxide trapped in the acid product. After that, the product was neutralized by 50% NaOH solution to pH 7, and diluted with deionized water to a desired concentration.

In one typical synthesis, the final product was collected as clear solution with pale yellow color. The product had a solid content of 60.29%. The successful sulfation was revealed by a Hyamine titration, from which the content of the anionic surfactant in product solution was determined as 57.44%.

### Example 2: Styrene-Butyl Acrylate Emulsion Polymerization

In the following examples, the emulsion polymerization reaction was performed in a 1 L glass flask reactor, which was equipped with thermometer, mechanical stirrer and condenser.

The monomer mixture composed of 148.4 g S, 120.4 g BA, 7.0 g MAA and 4.2 g AM was mixed with 126 g of water, 1.4 g of Anionic Surfactant 1, 2.96 g of RHODASURF® BC-8509, and emulsified by vigorous stirring to prepare a pre-monomer emulsion (PME). 120.0 g de-ionized water, 2.33 g of Anionic Surfactant 1 and 1.4 g SIPOMER® COPS-1 were charged into the reactor. The reactor contents were heated to 83-85° C. under nitrogen; then, added the solution of 0.56 g APS in 12.0 g water and 5% of the as prepared PME into the reactor. Maintained the reaction for 15 minute to allow for seed formation. After that, started drop-wise feeding of PME with the solution of 1.68 g APS in 50.0 g water during 2-3 h. After completion of the feeding, kept at the reactor temperature at 85° C. for 1 h. Cooled down the reactor to 75° C., and charged 0.4 g TBP and the solution of 0.4 g IAA in 1.4 g water, then maintained the reactor temperature at 75° C. for 30 minute. After cooling down to 30° C., the polymer emulsion was neutralized to pH 9-10 by 25% aqueous ammonia solution.

The conversion rate of the emulsion polymerization was 99.6%.

The conversion rate is defined as the ratio of the actual solid content to the designed solid content of the polymer emulsion obtained from the emulsion polymerization process.

### Example 3: Styrene-Butyl Acrylate Emulsion Polymerization

Steps same as those in Example 2 were conducted, except that Anionic Surfactant 1 was replaced by Anionic Surfactant 2. The conversion rate of the emulsion polymerization was 99.4%.

### Example 4: Full Acrylate Emulsion Polymerization

The procedure was same as Example 2, but the monomer mixture was composed of 136.4 g MMA, 140.4 g BA and 4.2 g MAA. The conversion rate of the emulsion polymerization was 99.7%.

### Example 5: Vinyl Acrylate Emulsion Polymerization

The reaction set-up was same as that in Example 2. The monomer mixture composed of 237.5 g VA, 42.2 g BA and 3.4 g AM was combined with 53.4 g of water, 2.8 g of Anionic Surfactant 1, 2.0 g of ABEX® AP-470, and emulsified with vigorous stirring to prepare a pre-monomer emulsion (PME). 164.1 g water, 1.2 g of Anionic Surfactant 2 and 2.8 g RHODASURF® BC8509 were charged into the reactor. The reactor contents were heated to 74-76° C. under nitrogen; then, added the solution of 0.26 g ferrous sulfate heptahydrate in 2.0 g water, as well as the solution of 0.63 g SPS and 0.11 g sodium acetate in 8.0 g water. Then started the drop-wise feeding of the PME with the solution of 0.63 g SPS in 12.0 g water and the solution of 0.13 g IAA and 0.65 g sodium acetate in 11.9 g water during 3 h. The reactor temperature was maintained at 74-76° C. After the completion of the feeding, cooled down the reactor to 65° C., then added the solution of 0.43 g TBP in 5.0 g water, 0.33 g sodium hydrosulfite in 5.0 g water and 0.12 g sodium acetate in 3.1 g water. Maintained the reactor temperature at 65° C. for 30 minute, and then cooled the reactor down to 40° C. The collected polymer emulsion was adjusted to pH 4-5 with sodium acetate aqueous solution. The conversion rate of the emulsion polymerization was 98.5%.

### Example 6: Physical Properties of the Emulsions

Styrene-butyl acrylate polymer emulsion was prepared following the procedures as described in Example 2, but the type and dosage of the anionic surfactants used varied and were summarized in Table 1 below. The quantity of the surfactant in Table 1 is the active weight of the surfactant, based on total monomer (BOTM).

TABLE 1

| Polymer | Anionic Surfactant in Kettle | Anionic Surfactant in PME |
|---|---|---|
| Emulsion | (% BOTM) | (% BOTM) |
| S1 | Anionic Surfactant 1: 0.2 | Anionic Surfactant 1: 0.4 |
| S2 | Anionic Surfactant 1: 0.5 | Anionic Surfactant 1: 0.3 |
| S3 | Anionic Surfactant 2: 0.5 | Anionic Surfactant 2: 0.3 |
| CS1 | RHODAPEX ® LA-40S: 0.2 | RHODAPEX ® LA-40S: 0.4 |

The obtained styrene-butyl acrylate polymer emulsions were characterized, and the physical properties were summarized in Table 2 below:

TABLE 2

| Polymer | Solids | Particle size | | Viscosity |
|---|---|---|---|---|
| Emulsion | (%) | (nm) | pH | (cP) |
| S1 | 47.55 | 152.1 | 9.01 | 600 |
| S2 | 47.62 | 120.4 | 9.01 | 1500 |
| S3 | 48.03 | 122.9 | 9.01 | 1520 |
| CS1 | 48.21 | 119.4 | 9.02 | 1680 |

In Table 2, the particle size was determined by Malvern Zetasizer Nano-ZS90; Viscosity was determined using Brookfield DV-IIPro Viscometer at 10 rpm with S3 spindle; Solid content was determined by weight loss after 2 hour at 120° C.

### Example 7: Foaming Property

The foaming property of the polymer emulsions prepared as described in Example 6 was studied. The emulsion was diluted to 10% by water. 100 g of the diluted emulsion were poured into a 250 ml bottle, and then agitated vigorously at 2000 rpm for 1 minute. The foaming height was recorded and the relative foaming height was calculated (the foaming height of CS1 group was set as 100%).

TABLE 3

| Polymer | |
|---|---|
| Emulsion | Relative Foaming Height |
| S1 | 70.7% |
| S2 | 80.5% |
| S3 | 78.5% |
| CS1 | 100% |

It can be seen that the inventive alkyl alkoxylate sulfates led to reduced foaming height compared to RHODAPEX® LA-40S.

### Example 8: Evaluation of Freeze-Thaw Stability

The freeze-thaw stability of the polymer emulsions prepared according to Example 6 was studied. 50 g of polymer emulsion were placed in −18° C. refrigerator for 16 hour to let it freeze. The sample was then placed at 25° C. for 8 hour to allow for thawing. The test was conducted for 5 cycles. It was observed that the polymer emulsion prepared by using RHODAPEX® LA-40S gelled at the first freeze-thaw cycle. In contrast, the polymer emulsions prepared by the inventive alkyl alkoxylate sulfates passed 5 cycles of freeze-thaw. It demonstrates that the inventive alkyl alkoxylate sulfates could improve the freeze-thaw stability of the polymer emulsion compared to RHODAPEX® LA-40S.

### Example 9: Evaluation of Freeze-Thaw Stability

Anionic Surfactant 3 was prepared according to the procedure known to a skilled person. Briefly, lauryl alcohol was firstly propoxylated, followed by ethoxylation. Then, the intermediate was subject to sulfation and subsequently neutralized with sodium hydroxide.

Polymer emulsions were prepared according to procedures as described in Example 2 and according to formulations in Table 4 below:

TABLE 4

| Polymer | Anionic Surfactant in Kettle | Anionic Surfactant in PME |
|---|---|---|
| Emulsion | (% BOTM) | (% BOTM) |
| S4 | Anionic Surfactant 1: 0.2 | Anionic Surfactant 1: 0.4 |
| S5 | Anionic Surfactant 2: 0.5 | Anionic Surfactant 2: 0.3 |
| CS2 | Anionic Surfactant 3: 0.2 | Anionic Surfactant 3: 0.4 |

50 g of polymer emulsion were placed in −18° C. refrigerator for 16 hour to let it freeze. The sample was then placed at 25° C. for 8 hour to allow for thawing. The test was conducted for 5 cycles. The viscosity of the emulsion was measured after each freeze thaw cycle, by using Brookfield DV-IIPro Viscometer at 60 rpm with S3 spindle. Results are shown in Table 5 below:

TABLE 5

| | Viscosity (cP) | | | | |
|---|---|---|---|---|---|
| | Cycle 1 | Cycle 2 | Cycle 3 | Cycle 4 | Cycle 5 |
| S4 | 920 | 1894 | 1493 | 1763 | 2010 |
| S5 | 730 | 1430 | 1567 | 1600 | 1680 |
| CS2 | 3037 | 3207 | 3267 | 3363 | 3347 |

Results showed that the emulsion prepared by using Anionic Surfactant 3 exhibited high viscosity after freeze thaw, indicating gelling and/or segregation of the emulsion. In contrast, the emulsion prepared by using the inventive anionic surfactants showed markedly lower viscosity after the freeze-thaw cycles and the emulsion remained stable, as well as fluid.

### Example 10: Evaluation of Ca²⁺ Stability

The Ca²⁺ stability of the polymer emulsions prepared according to Example 6 was studied. 30 ml of polymer emulsion were added into a beaker, and then added 6 ml of 0.5% CaCl₂ aqueous solution. Mixed them well, and kept the solution at ambient temperature for 48 hour. The Ca²⁺ stability test was regarded as pass if there was no phase separation, precipitate formation or gel formation observed after standing for 48 hour.

All the polymer emulsion samples prepared according to Example 6 passed the Ca²⁺ stability test.

1-15: (canceled)

16. A compound of formula (I):
R₁O—(CH₂CH₂O)ₓ—(CH₂CH(R₂)—O)_{y}—SO₃M (I)
wherein R₁ is a linear or branched, saturated or unsaturated, C₄-C₁₈ hydrocarbon group;
R₂ is CH₃ or CH₂CH₃;
x is a real number in the range of from 1 to 11;
y is a real number in the range of from 1 to 20; and
M is a cation.

17. The compound according to claim 16, wherein R₁ is a linear or branched C₄-C₁₈ alkyl group.

18. The compound according to claim 16, wherein R₁ is a linear or branched C₁₂-C₁₈ alkyl group.

19. The compound according to claim 16, wherein x is a real number in the range of from 3 to 8.

20. The compound according to claim 16, wherein y is a real number in the range of from 3 to 8.

21. The compound according to claim 16, wherein M is selected from the group consisting of Na⁺, K⁺, Li⁺ and —(HNR₃R₄R₅)⁺ wherein R₃, R₄ and R₅ are independently H, a C₁-C₆ alkyl or C₁-C₆ hydroxylalkyl group.

22. A composition comprising the compound according to claim 16.

23. The composition according to claim 22, wherein the composition further comprises a compound of formula (II):
R₁O—(CH₂CH₂O)ₓ—(CH₂CH(R₂)—O)_{y}—H (II)
wherein R₁ is a linear or branched, saturated or unsaturated, C₄-C₁₈ hydrocarbon group;
R₂ is CH₃ or CH₂CH₃;
x is a real number in the range of from 1 to 11;
y is a real number in the range of from 1 to 20.

24. The composition according to claim 23, wherein the weight ratio of said compound of formula (I) to said compound of formula (II) is in the range of from 90:10 to 70:30.

25. The composition according to claim 22, wherein the composition further comprises a co-surfactant.

26. The composition according to claim 25, wherein the co-surfactant is an ethoxylated alcohol or a salt thereof.

27. The composition according to claim 22, wherein the composition further comprises water.

28. The composition according to claim 22, wherein said compound of formula (I) is present in an amount of from 30 wt % to 60 wt % based on the total weight of the composition.

29. A method for the emulsion polymerization of at least one ethylentically unsaturated monomer comprising at least one carbon-to-carbon double bond, said method comprising polymerizing said ethylentically unsaturated monomer in an aqueous medium in the presence of the compound according to claim 16, and a water-soluble initiator.

30. A method for the emulsion polymerization of at least one ethylentically unsaturated monomer comprising at least one carbon-to-carbon double bond, said method comprising polymerizing said ethylentically unsaturated monomer in an aqueous medium in the presence of the composition according to claim 22, and a water-soluble initiator.
